SEQUENCE LISTING (12 sequences)

SEQ ID NO: 1 - protein, 447 aa, Artificial Sequence
  Description of Artificial Sequence Synthetic polypeptide
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 215 aa, Artificial Sequence
  Description of Artificial Sequence Synthetic polypeptide
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 5 aa, Artificial Sequence
  Description of Artificial Sequence Synthetic peptide
SNVIS

SEQ ID NO: 4 - protein, 17 aa, Artificial Sequence
  Description of Artificial Sequence Synthetic peptide
GVIPIVDIANYAQRFKG

SEQ ID NO: 5 - protein, 11 aa, Artificial Sequence
  Description of Artificial Sequence Synthetic peptide
TLGLVLDAMDY

SEQ ID NO: 6 - protein, 12 aa, Artificial Sequence
  Description of Artificial Sequence Synthetic peptide
RASQSLGSSYLA

SEQ ID NO: 7 - protein, 7 aa, Artificial Sequence
  Description of Artificial Sequence Synthetic peptide
GASSRAP

SEQ ID NO: 8 - protein, 9 aa, Artificial Sequence
  Description of Artificial Sequence Synthetic peptide
QQYADSPIT

SEQ ID NO: 9 - protein, 444 aa, Artificial Sequence
  Description of Artificial Sequence Synthetic polypeptide
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial Sequence
  Description of Artificial Sequence Synthetic polypeptide
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 448 aa, Artificial Sequence
  Description of Artificial Sequence Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0036928 A1
(43) Pub. Date: Feb. 2, 2023

(54) ANTI-TGF-BETA ANTIBODY FORMULATIONS AND THEIR USE

(71) Applicant: Genzyme Corporation, (US)

(72) Inventors: Kiran Rana BANGARI, Framingham, MA (US); Ramil LATYPOV, Wellesley, MA (US); Timothy MCCOY, Shrewsbury, MA (US); Sanket PATKE, Cambridge, MA (US)

(73) Assignee: Genzyme Corporation, Cambridge (US)

(21) Appl. No.: 17/843,812

(22) Filed: Jun. 17, 2022

Related U.S. Application Data
(60) Provisional application No. 63/212,473, filed on Jun. 18, 2021.

(51) Int. Cl. C07K 16/22; A61K 9/08; A61K 47/26; A61K 47/12; A61K 45/06; A61K 47/20; A61K 47/22; A61K 9/00; A61K 47/18

(52) U.S. Cl. CPC C07K 16/22; A61K 9/08; A61K 47/26; A61K 47/12; A61K 45/06; A61K 47/20; A61K 47/22; A61K 9/0019; A61K 47/183

(57) ABSTRACT

The present disclosure provides pharmaceutical compositions comprising anti-TGF-β antibodies and methods of use thereof.

### CROSS REFERENCE TO RELATED APPLICATION

This application claims priority from U.S. Provisional Application 63/212,473, filed on Jun. 18, 2021. The disclosure of this priority application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 17, 2022, is named 022548 US064 SL.txt and is 19,437 bytes in size.

### BACKGROUND OF THE INVENTION

Transforming growth factor beta (TGF-β) is a cytokine that controls many key cellular functions including proliferation, differentiation, survival, migration, and epithelial mesenchymal transition. It regulates diverse biologic processes, such as extracellular matrix formation, wound healing, embryonic development, bone development, hematopoiesis, immune and inflammatory responses, and malignant transformation. Deregulation of TGF-β leads to pathological conditions, e.g., birth defects, cancer, chronic inflammation, and autoimmune and fibrotic diseases.

TGF-β has three known isoforms—TGF-β1, 2, and 3. All three isoforms are initially translated as a pro-peptide. After cleavage, the mature C-terminal end remains associated with the N-terminus (called the latency associated peptide or LAP), forming the small latent complex (SLC), which is secreted from the cell. The inability of the SLC to bind to TGF-β receptor II (TGFβRII) prevents receptor engagement. Activation by dissociation of the N- and C-termini occurs by one of several mechanisms, including proteolytic cleavage, acidic pH, or integrin structural alterations (Connolly et al., Int J Biol Sci. (2012) 8(7):964-78).

TGF-β1, 2, and 3 are pleiotropic in their function and expressed in different patterns across cell and tissue types. They have similar in vitro activities, but individual knockouts in specific cell types suggest non-identical roles in vivo despite their shared ability to bind to the same receptor (Akhurst et al., Nat Rev Drug Discov. (2012) 11(10):790-811). Upon TGF-β binding to TGFβRII, the constitutive kinase activity of the receptor phosphorylates and activates TGF-β receptor I (TGFβRI), which phosphorylates SMAD2/3, allowing for association to SMAD4, localization to the nucleus, and transcription of TGF-β-responsive genes. Id. In addition to this canonical signaling cascade, a non-canonical pathway transmits signals through other factors including p38 MAPK, PI3K, AKT, JUN, JNK, and NF-κB. TGF-β signaling is also modulated by other pathways, including WNT, Hedgehog, Notch, INF, TNF, and RAS. Thus, the end result of TGF-β signaling is a crosstalk of all of these signaling pathways that integrates the state and environment of the cell. Id.

Given the diverse functions of TGF-β, there is a need for efficacious pan-TGF-β-specific antibody therapy.

### SUMMARY OF THE INVENTION

The present disclosure provides pharmaceutical compositions that comprise an anti-TGF-β antibody. In one aspect, the disclosure provides a pharmaceutical composition, wherein the composition is an aqueous liquid solution comprising: 20-200 mg/ml anti-TGFβ antibody, wherein the antibody comprises a heavy chain variable domain (V_{H}) amino acid sequence corresponding to residues 1-120 of SEQ ID NO:1 and a light chain variable domain (V_{L}) amino acid sequence corresponding to residues 1-108 of SEQ ID NO:2, 10-50 mM acetate, optionally 25 mM acetate, and 5-15% w/v sucrose, optionally 8% w/v sucrose, wherein the solution has a pH of 5.0±0.2 or 5.0±0.3. In some embodiments, the composition is an aqueous liquid solution with a pH of 4.7-5.3.

In some embodiments, the antibody comprises a heavy chain amino acid sequence set forth in SEQ ID NO:1 (with or without the C-terminal lysine) and a light chain amino acid sequence set forth in SEQ ID NO:2.

In some embodiments, the anti-TGFβ antibody is at a concentration of 40-180 mg/ml, optionally 50 mg/ml or 150 mg/ml.

In some embodiments, the composition comprises a surfactant, such as polysorbate (e.g., polysorbate 80 (PS80)). In particular embodiments, the composition comprises PS80 at a concentration of 0.01-0.10% w/v, optionally 0.06% w/v.

In some embodiments, the composition comprises a chelating agent, optionally selected from EDTA and DPTA. In certain embodiments, the chelating agent is at a concentration of 0 to 20 μM, optionally 10 μM.

In particular embodiments, the composition comprises 50 mg/ml, 75 mg/ml, or 150 mg/ml anti-TGFβ antibody, 25 mM acetate, 10 μM EDTA, 0.06% PS80, and 8% w/v sucrose, with a pH of 5.0±0.3. In certain embodiments, the antibody comprises a heavy chain amino acid sequence set forth in SEQ ID NO:1 (with or without the C-terminal lysine) and a light chain amino acid sequence set forth in SEQ ID NO:2.

The present disclosure also provides an article of manufacture, comprising a vial and instructions for use, wherein the vial contains about 16 ml of the present composition.

Also provided herein are methods of treating cancer in a patient in need thereof, comprising administering to the patient a therapeutically effective amount of the present composition. In some embodiments, the method further comprises administering an additional anti-cancer therapeutic. In particular embodiments, the composition is administered intravenously at a dose of 5 mg/kg or 15 mg/kg, optionally biweekly. The present disclosure also provides the present the composition for use in treating a patient in need thereof in these methods, and the use of the present composition for the manufacture of a medicament for treating a patient in need thereof in a treatment method of the present disclosure.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a photograph showing the degree of opalescence in acetate or histidine buffer formulations of 80 mg/ml Ab1 at a range of pH.

FIGS. 2A-C are panels of bar graphs showing the evolution of 2 μm (FIG. 2A), 10 μm (FIG. 2B), and 25 μm (FIG. 2C) subvisible particles in acetate and histidine formulations over 4 weeks of storage at 5° C., 25° C., or 40° C.

FIG. 3 is a bar graph showing the viscosity values (in centipoise (cP)) of acetate and histidine formulations immediately after preparation (T₀) and after four weeks of storage at 40° C.

FIGS. 4A and 4B are panels of bar graphs showing the pH values of acetate and histidine formulations over 4 weeks of storage at 40° C., 25° C., or 5° C.

FIG. 5 is a scatter plot graph showing optical density values (340-360 nm) of the acetate (pH 4.7, 5, and 5.5) and histidine (pH 5.5, 6, and 6.5) at T₀ and after 4 weeks of storage at 5° C., 25° C., and 40° C.

FIG. 6 is a panel of graphs showing the HMWS evolution in formulations at different polysorbate (PS80) concentrations over 2 weeks of storage at 5° C. (top-left panel), 2 weeks of storage at 40° C. (top-right panel), rigorous agitation for 48 hours (bottom-left panel), and freeze/thaw (FT) cycling at −30° C. to room temperature (bottom-right panel). SR_# or Ch_#: the # is the % concentration of PS80. SR and Ch represent two different vendors for PS80.

FIG. 7A is a pair of bar graphs showing Ab1 concentrations after dilution into saline or dextrose in polyolefin (PO) or polyvinyl chloride (PVC) IV bags.

FIG. 7B is a pair of bar graphs showing subvisible (≥10 pin) particles after dilution into saline (S) or dextrose (D) in PO or PVC IV bags. T0: time zero. T24: 24 hours. T48: 48 hours.

FIGS. 8A and 8B are graphs showing HMWS evolution in various concentrations of Ab1 formulations over 12 weeks of storage at 5° C., 25° C., and 40° C. (FIG. 8A) and over 6 months of storage at −20° C. (FIG. 8B).

FIG. 9 is a pair of graphs showing M252 oxidation (left panel) and HMWS % (right panel) evolution in metal-spiked Ab1 formulations.

FIG. 10 is a pair of bar graphs showing HMWS and subspecies evolution in various formulations after one month of storage at 40° C. (left panel) or three months of storage at 25° C.

### DETAILED DESCRIPTION OF THE INVENTION

The present disclosure provides stable pharmaceutical compositions comprising a pan-TGFβ-specific monoclonal antibody in an aqueous liquid solution. One such antibody is Ab1. Ab1 is an IgG₄ monoclonal antibody that targets all three isoforms of human TGF-β (TGF-β1, TGF-β2, and TGF-β3) and has a heavy chain amino acid sequence of SEQ ID NO:1 and a light chain amino acid sequence of SEQ ID NO:2.

Therapeutic success of monoclonal antibodies depends in part on the antibody drug candidates' manufacturability, stability, and delivery attributes. Poor solution behavior, e.g., high solution viscosity or opalescence, profoundly impacts the developability of antibody drugs. Formulation studies on Ab1 have shown that this antibody is surface active and has a high propensity of forming subvisible and visible particulates upon solution agitation or under other interfacial stress conditions. The inventors have discovered that the present formulations, which are based on an acetate buffer having an acidic pH around 5.0, significantly improved the stability of the formulation during storage and transportation, including reducing particulate formation. The inventors have discovered that Ab1 displays undesirable solution behavior such as opalescence and poor colloidal stability at a higher pH (e.g., pH 6.0). The inventors have also discovered that particle formation in solution can be further mitigated by the addition of a surfactant and that inclusion of a chelator agent also helps improve the formulation.

### I. Pan-Specific Anti-TGFβ Monoclonal Antibodies

The monoclonal antibody formulated herein comprise the complementarity-determining regions (CDRs) in Ab1. Such antibodies are collectively termed “Ab1-related antibodies” herein, which include Ab1 itself. In some embodiments, the antibody is a fully human antibody comprising a human IgG₄ constant region and a human κ light chain constant region. In further embodiments (e.g., Ab1), the human IgG₄ constant region has a mutation at position 228 (Eu numbering). In some embodiments (e.g., Ab1), the mutation is a serine-to-proline mutation (S228P).

The heavy and light chain amino acid sequences of Ab1 are shown below as SEQ ID NOs:1 and 2, respectively. The S228P site is in box and boldface in the sequence of SEQ ID NO:1. Variable domains are italicized. CDRs are shown in boxes. The glycosylation site in the constant domain of the heavy chain is in boldface (N297).

(SEQ ID NO: 1)
QVQLVQSGAE VKKPGSSVKV SCKASGYTFS

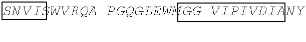

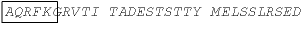

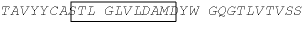

ASTKGPSVFP LAPCSRSTSE STAALGCLVK
DYFPEPVTVS WNSGALTSG HTFPAVLQSS
GLYSLSSVVT VPSSSLGTKT YTCNVDHKPS

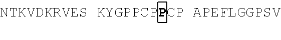

FLFPPKPKDT LMISRTPEVT CVVVDVSQED
PEVQFNWYVD GVEVHNAKTK PREEQFNSTY
RVVSVLTVLH QDWLNGKEYK CKVSNKGLPS
SIEKTISKAK GQPREPQVYT LPPSQEEMTK
NQVSLTCLVK GFYPSDIAVE WESNGQPENN
YKTTPPVLDS DGSFFLYSRL TVDKSRWQEG
NVFSCSVMHE ALHNHYTQKS LSLSLGK
(SEQ ID NO: 2)

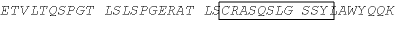

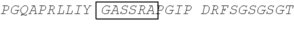

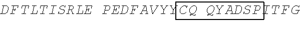

QGTRLEIKRT VAAPSVFIFP PSDEQLKSGT
ASVVCLLNNF YPREAKVQWK VDNALQSGNS
QESVTEQDSK DSTYSLSSTL TLSKADYEKH
KVYACEVTHQ GLSSPVTKSF NRGEC

In some embodiments, the antibody herein has an antibody (e.g., a human antibody) having the CDRs shown above. That is, the antibodies have the following heavy chain and light chain CDR amino acid sequences:

HCDR1
(SEQ ID NO: 3)
SNVIS
HCDR2
(SEQ ID NO: 4)
GVIPIVDIANYAQRFKG
HCDR3
(SEQ ID NO: 5)
TLGLVLDAMDY
LCDR1
(SEQ ID NO: 6)
RASQSLG SSYLA
LCDR2
(SEQ ID NO: 7)
GASSRAP
LCDR3
(SEQ ID NO: 8)
QQYADSPIT

Thus, the antibody may comprise SEQ ID NOs:3, 4, 5, 6, 7, and 8.

In further embodiments, the antibody has a heavy chain variable domain (amino acids 1-120 of SEQ ID NO:1) and a light chain variable domain (amino acids 1-108 of SEQ ID NO:2) shown above. In certain embodiments, the antibody formulated herein does not have the C-terminal lysine in the heavy chain.

In particular embodiments, the antibody formulated herein is Ab1. Ab1 has an estimated molecular weight of 144 kDa when un-glycosylated. Ab1 has a molecular weight of 147.011 kDa as determined by mass spectrometry, a theoretical experimental isoelectric point (pI) of 6.78, and an experimental pI of about 5.9-7.1.

### II. Methods of Making Antibodies

Ab1-related antibodies can be made by methods well established in the art. DNA sequences encoding the heavy and light chains of the antibodies can be inserted into expression vectors such that the genes are operatively linked to necessary expression control sequences such as transcriptional and translational control sequences. Expression vectors include plasmids, retroviruses, adenoviruses, adeno-associated viruses (AAV), plant viruses such as cauliflower mosaic virus, tobacco mosaic virus, cosmids, YACs, EBV derived episomes, and the like. The antibody light chain coding sequence and the antibody heavy chain coding sequence can be inserted into separate vectors, and may be operatively linked to the same or different expression control sequences (e.g., promoters). In one embodiment, both coding sequences are inserted into the same expression vector and may be operatively linked to the same expression control sequences (e.g., a common promoter), to separate identical expression control sequences (e.g., promoters), or to different expression control sequences (e.g., promoters). The antibody coding sequences may be inserted into the expression vector by standard methods (e.g., ligation of complementary restriction sites on the antibody gene fragment and vector, or blunt end ligation if no restriction sites are present).

In addition to the antibody chain genes, the recombinant expression vectors may carry regulatory sequences that control the expression of the antibody chain genes in a host cell. Examples of regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters.

In addition to the antibody chain genes and regulatory sequences, the recombinant expression vectors of the invention may carry additional sequences, such as sequences that regulate replication of the vector in host cells (e.g., origins of replication) and selectable marker genes. For example, the selectable marker gene confers resistance to drugs, such as G418, hygromycin or methotrexate, on a host cell into which the vector has been introduced. Selectable marker genes may include the dihydrofolate reductase (DHFR) gene (for use in dhfr-host cells with methotrexate selection/amplification), the neo gene (for G418 selection), and the glutamate synthetase gene.

The expression vectors encoding the antibodies of the present disclosure are introduced to host cells for expression. The host cells are cultured under conditions suitable for expression of the antibody, which is then harvested and isolated. Host cells include mammalian, plant, bacterial or yeast host cell. Mammalian cell lines available as hosts for expression are well known in the art and include many immortalized cell lines available from the American Type Culture Collection (ATCC). These include, inter alia, Chinese hamster ovary (CHO) cells, NS0 cells, SP2 cells, HEK-293T cells, 293 Freestyle cells (Invitrogen), NIH-3T3 cells, HeLa cells, baby hamster kidney (BHK) cells, African green monkey kidney cells (COS), human hepatocellular carcinoma cells (e.g., Hep G2), A549 cells, and a number of other cell lines. Cell lines may be selected based on their expression levels. Other cell lines that may be used are insect cell lines, such as Sf9 or Sf21 cells.

Further, expression of antibodies can be enhanced using a number of known techniques. For example, the glutamine synthetase gene expression system (the GS system) is a common approach for enhancing expression under certain conditions.

Tissue culture media for the host cells may include, or be free of, animal-derived components (ADC), such as bovine serum albumin. In some embodiments, ADC-free culture media is preferred for human safety. Tissue culture can be performed using the fed-batch method, a continuous perfusion method, or any other method appropriate for the host cells and the desired yield.

### III. Antibody Formulations

The present formulations confer superior stability to Ab1-related anti-TGF-β antibodies, including Ab1. “Stable” or “stability” refers to the ability of the antibody in a composition to retain its physical stability, chemical stability, and/or biological activity during storage, and/or when subjected to physical or chemical stress. Stability can be in the context of a selected temperature, for example, under frozen conditions (e.g., −70° C. to −30° C.), under refrigerated conditions (e.g., 2-8° C.), or at room temperature (e.g., 23-25° C.), for a selected time period, e.g., 16 weeks, 24 weeks, 36 weeks, four months, six months, one year, two years, three years, or longer. Stability of a protein may be measured in assays that are conducted within a shorter period of time but whose results are indicative of stability in clinical settings. Such assays include freeze/thaw cycling assays where a protein composition is subjected to one or more freeze-thaw cycles; or agitation assays where a protein composition is subjected to mechanic agitation treatment over a pre-determined period. Protein stability may be determined by storing the protein composition at a designated storage temperature (such as 2-8° C.) over a selected time period and analyzing its structural and functional attributes, such as degree of dimerization or aggregation (e.g., as measured by size exclusion HPLC or protein gel), protein degradation (e.g., as measured by size exclusion HPLC or protein gel), color change of the composition, clarity of a liquid composition, enzymatic activity, glycan content and composition, receptor binding affinity, methionine residual oxidation, and the biological activity of the composition. See also the Examples below for more detailed illustration of methodology for examining stability of an antibody formulation.

An antibody described herein “retains its chemical stability” in a pharmaceutical composition, if the chemical stability at a given time is such that the antibody is considered to maintain its biological activity as defined below. To assess chemical stability, chemically altered forms of the antibody may be detected and quantified. Chemical alteration may involve size modification and can be evaluated using methods known in the art such as size exclusion chromatography, capillary isoelectric focusing (cIEF), liquid chromatography/mass spectrometry (LCMS), SDS-PAGE, and/or matrix-assisted laser desorption ionization/time-of-flight mass spectrometry (MALDI/TOF MS). Other types of chemical alteration include charge alteration, which, for example, may occur as a result of deamidation or oxidation, and can be evaluated by ion-exchange chromatography, mass spectrometry, or size exclusion chromatography. In some embodiments, a type of chemical alteration that occurs during accelerated storage of the compositions comprising the antibodies described herein, involves the oxidation of the antibodies. In some embodiments, residues M252 and M428 of SEQ ID NO:1 are oxidized in metal-spiked samples at accelerated storage.

The compositions of the present disclosure contain one or more pharmaceutically acceptable excipients. The term “excipient” or “carrier” is used herein to describe any ingredient other than the compound(s) of the invention. An excipient may be an inert substance that is used as a diluent, vehicle, carrier, preservative, binder, or stabilizing agent for the active ingredient(s) of a drug. For example, the compositions may contain a buffering agent, an isotonic agent, and/or a stabilizing agent such as an anti-oxidant. In some cases, one agent may serve more than one of these purposes. In some embodiments, a composition of the invention contains an anti-TGF-β antibody described herein, a buffering agent such as acetate, a stabilizer such as sucrose, and a surfactant such as polysorbate 80 (PS80). The anti-TGF-β antibody described herein has improved stability due to the combination of specific components in the composition. The compositions of the invention may be aqueous liquid solutions or lyophilized preparations. In preferred embodiments, the compositions of the invention are liquid aqueous solutions.

In some embodiments, the composition comprises a stabilizing agent such a L-methionine. In particular embodiments, the composition is an aqueous liquid composition comprising 5-20 mM (e.g., 10 mM) L-methionine.

In some embodiments, the composition comprises a bulking agent such as mannitol. In particular embodiments, the composition is an aqueous liquid composition comprising 1-10% (e.g., 3.5%) mannitol (w/v).

In some embodiments, the composition is an aqueous liquid composition comprising a buffer such as an L-histidine buffer. In particular embodiments, the aqueous liquid composition comprises 5-20 mM (e.g., 10 mM) L-histidine.

In some embodiments, the pH of the buffer ranges from about 4.0 to about 6.0. In some embodiments, the pH of the buffer is 6.0. In a preferred embodiment, the pH of the buffer is 5.0±0.3. In some embodiments, the pH of the buffer is adjusted with sodium hydroxide.

In some embodiments, the composition is an aqueous liquid composition comprising 40-180 mg/ml (e.g., 50-150 mg/ml) an Ab1-related antibody (e.g., Ab1); 10-50 mM (e.g., 10-30 mM) acetate; and 1-10% (e.g., 6-8%) w/v sucrose. In some other embodiments, the composition is an aqueous liquid composition comprising 15-40 mg/mL anti-TGF-β monoclonal antibodies, 5-20 mM (e.g., 10 mM) L-histidine, 1-10% (e.g., 6-8%) sucrose (w/v), 1-10% (e.g., 3.5%) mannitol (w/v), and 5-20 mM (10 mM) L-methionine. The pH of the aqueous liquid composition may be 4.0-6.0 (e.g., 4.7-5.5).

In some embodiments, the aqueous liquid composition comprises 0.01-0.07% w/v surfactant(s). Exemplary surfactants include nonionic detergents, such as polysorbates (e.g., polysorbates 20 and 80) and poloxamers (e.g., poloxamer 188). In some embodiments, the aqueous liquid composition comprises 0.01-0.07% polysorbate 80 (e.g., more than 0.025% or 0.05-0.06% PS80). In some cases, the presence of surfactant(s) may help to reduce turbidity/opalescence in the liquid composition.

In some embodiments, the aqueous liquid composition comprises 0 to 50 μM (e.g., 10 μM) chelating agent(s), such as EDTA or DPTA.

In a preferred embodiment, the composition is an aqueous liquid composition comprising 50 or 150 mg/ml anti-TGF-β monoclonal antibodies, 25 mM acetate, 10 μM EDTA or DPTA, 0.06% PS80, and 8% w/v sucrose. In particular embodiments, the aqueous liquid composition has a pH of 5±0.3.

In some embodiments, the composition is an aqueous liquid composition comprising 25 mg/mL anti-TGF-β monoclonal antibodies, 10 mM L-histidine, 2% (w/v) sucrose, 3.5% (w/v) mannitol, 10 mM L-methionine, 0.01% (w/v) polysorbate 80, with the aqueous liquid composition having a pH of 6.0. In particular embodiments, the composition is an aqueous liquid composition comprising 25 mg/mL anti-TGF-β monoclonal antibodies, 1.18 mg/mL L-histidine monohydrochloride, 0.68 mg/mL L-histidine, 1.5 mg/mL L-methionine, 0.1 mg/mL polysorbate 80, 20 mg/mL sucrose, and 35.3 mg/mL mannitol at a pH of 6.0.

| Ingredients | Conc. (mg/mL) |
|---|---|
| Ab1 | 25.0 |
| L-histidine monohydrochloride | 1.18 |
| L-histidine | 0.68 |
| L-methionine | 1.50 |
| Polysorbate 80 | 0.10 |
| Sucrose | 20.0 |
| Mannitol | 35.3 |
| Water for Injection | q.s. |

The aqueous liquid compositions may be prepared by mixing Ab1 produced by recombinant technology and subsequently purified from host cells, with excipients described herein in water, and adjusting the resulting mixture to the desired pH. For example, the anti-TGF-β monoclonal antibodies and desired excipients may be added to, or buffer-exchanged into, acetate buffer with the desired pH.

In some embodiments, the aqueous liquid composition may be prepared by reconstituting a lyophilized composition of the invention. The reconstitution may be done with a pharmaceutically acceptable liquid such as sterile water, saline (e.g., 0.9% sodium chloride), or acetate-buffered saline.

### IV. Articles of Manufacture

The compositions of the invention may be supplied in an article of manufacture (e.g., a kit) that includes instructions for use and optionally other therapeutic agents for treating disorders. The active pharmaceutical ingredient (API) in the articles (e.g., Ab1) may be supplied in an amount that can be readily administered in accordance to the dosing regimens described herein.

For example, the article of manufacture may include a vial that contains 800 mg of Ab1 in 16 mL of an aqueous liquid solution comprising 25 mM acetate, 8% sucrose, 10 μM EDTA or DTPA, 0.06% PS80, at pH 5.0±0.3. In some embodiments, the vial contains 800 mg of Ab1, 15 mg of acetate, 800 mg of sucrose, and 6 mg of PS80. In some embodiments, the vial contains 800 mg of Ab1, 24 mg of acetate, 1280 mg of sucrose, and 9.6 mg of PS80. In particular embodiments, the vial is a pre-treated glass vial containing a standard closure. For example, the vial may be an ISO 20R type 1 tubing glass vial with a West 20 mm stopper as the closure.

The present compositions may be stored at −3° C. to 5° C. for two or more years.

### V. Use of Ab1 and Related Antibodies

The TGF-β receptor is widely expressed on immune cells, leading to broad effects of TGF-β in both the innate and adaptive immune system. TGF-β has been linked to many diseased conditions, for example, birth defects, cancer, chronic inflammation, autoimmunity, and fibrotic diseases. A therapeutic amount of Ab1 or a related antibody may be used to treat these conditions. A “therapeutically effective” amount refers to the amount of Ab1, a related antibody, or another therapeutic agent referred to herein, that relieves one or more symptoms of the treated condition. This amount may vary based on the condition or patient being treated, and can be determined by a healthcare professional using well established principles.

The appropriate dosage level of the pharmaceutical composition described herein may be determined on the basis of a variety of factors, including the patient's age, weight, disease condition, general health, and medical history, as well as the route and frequency of the drug administration, the pharmacodynamics and pharmacokinetics of the Ab1 active ingredient in the drug, and any other drugs that the patient may be taking concurrently. In some embodiments, the Ab1 or related antibody may be administered at 40, 20, or 15 mg/kg or less (such as 14, 13, 12, 11, 10, 9, 8, 7, 6, 5, 4, 3, 2, or 1 mg/kg). In some embodiments, the Ab1 may be administered at 5 mg/kg and 15 mg/kg. The dosing frequency may be, for example, daily, every two, three, four, or five days, weekly, biweekly, or triweekly, monthly, or bimonthly. In some embodiments, the dosing frequency is biweekly. Intervals between successive doses may be two weeks, or shorter or longer than two weeks as determined to be appropriate by a clinician.

The antibody may be administered intravenously (e.g., intravenous infusion over 0.5-8 hours), subcutaneously, topically, or any other route of administration that is appropriate for the condition and the drug formulation.

Ab1 and related antibodies are derived from human antibody genes and thus have low immunogenicity in humans; however, patients may be monitored for adverse events when treating patients with Ab1 or a related antibody.

In some embodiments, efficacy of the antibodies of the invention can be indicated by one or more of the following in the patient (e.g., in an affected tissue such as tumor tissue in the patient): (1) a decrease in the level or activity of TGF-β, (2) an increase in MIP2 and/or KC/GRO levels, (3) activation or infiltration to the tumor tissue of CD8+ T cells such as INF-γ-positive CD8+ T cells, and (4) an increase in clustering of natural killer (NK) cells.

The patients may be adults (e.g., patients 18 years or older, including geriatric patients who are 65 years or older). The patients may be pediatric patients (patients who are younger than 18 years old, e.g., patients who are newborn to 6 years old, who are 6 to 12 years old, or who are 12 to 18 years old).

In certain embodiments, a pharmaceutical Ab1 composition containing Ab1 at 50 mg/ml and containing 25 mM Acetate, 8% sucrose, 10 μM EDTA or DPTA, and 0.06% PS80 (pH 5.0±0.3) (e.g., supplied in 10 mL vials) is administered intravenously to patients at 5 mg/kg or 15 mg/kg biweekly until such time when a desired therapeutic endpoint has been achieved. For IV administration, the Ab1 composition may be diluted in saline or IV dextrose fluid (typically containing 5% dextrose in water). PO or PVC IV bags, for example, may be used. In some embodiments, the Ab1 formulation is diluted in saline in PVC bags prior to use. In some embodiments, the Ab1 formulation is diluted in IV dextrose fluid in PVC bags prior to use. In some embodiments, the Ab1 formulation is diluted in saline in PO bags prior to use. In some embodiments, the Ab1 formulation is diluted in IV dextrose fluid in PO bags prior to use.

A. Non-Oncological Diseased Conditions

Conditions that can be treated by Ab1 and related antibodies may include, without limitation, bone defects (e.g., osteogenesis imperfecta), glomerulonephritis, neural or dermal scarring, lung or pulmonary fibrosis (e.g., idiopathic pulmonary fibrosis), radiation-induced fibrosis, hepatic fibrosis, myelofibrosis, scleroderma, immune-mediated diseases (including rheumatoid arthritis, multiple sclerosis, systemic lupus erythematosus, Sjogren's syndrome, Berger's disease, and transplant rejection), and Dupuytren's contracture.

They may also be useful for treating, preventing and reducing the risk of occurrence of renal insufficiencies, including but not limited to, focal segmental glomerulosclerosis (FSGS), diabetic (type I and type II) nephropathy, radiational nephropathy, obstructive nephropathy, diffuse systemic sclerosis, hereditary renal disease (e.g., polycystic kidney disease, medullary sponge kidney, horseshoe kidney), glomerulonephritis, nephrosclerosis, nephrocalcinosis, systemic or glomerular hypertension, tubulointerstitial nephropathy, renal tubular acidosis, renal tuberculosis, and renal infarction. In particular, they are useful when combined with antagonists of the renin-angiotensin-aldosterone system including but not limited to: renin inhibitors, angiotensin-converting enzyme (ACE) inhibitors, Ang II receptor antagonists (also known as “Ang II receptor blockers”), and aldosterone antagonists. See, e.g., WO 2004/098637, whose disclosure is incorporated by reference herein in its entirety.

Ab1 and related antibodies are useful to treat diseases and conditions associated with the deposition of ECM, such as systemic sclerosis, postoperative adhesions, keloid and hypertrophic scarring, proliferative vitreoretinopathy, glaucoma drainage surgery, corneal injury, cataract, Peyronie's disease, adult respiratory distress syndrome, cirrhosis of the liver, post myocardial infarction scarring, post angioplasty restenosis, scarring after subarachnoid hemorrhage, fibrosis after laminectomy, fibrosis after tendon and other repairs, biliary cirrhosis (including sclerosing cholangitis), pericarditis, pleurisy, tracheostomy, penetrating CNS injury, eosinophilic myalgic syndrome, vascular restenosis, veno-occlusive disease, pancreatitis and psoriatic arthropathy.

Ab1 and related antibodies further are useful in conditions where promotion of re-epithelialization is beneficial. Such conditions include but are not limited to diseases of the skin, such as venous ulcers, ischemic ulcers (pressure sores), diabetic ulcers, graft sites, graft donor sites, abrasions and burns, diseases of the bronchial epithelium, such as asthma, ARDS, diseases of the intestinal epithelium, such as mucositis associated with cytotoxic treatment, esophageal ulcers (reflex disease), gastro-esophageal reflux disease, stomach ulcers, small intestinal and large intestinal lesions (inflammatory bowel disease).

Still further uses of Ab1 and related antibodies are in conditions in which endothelial cell proliferation is desirable, for example, in stabilizing atherosclerotic plaques, promoting healing of vascular anastomoses, or in conditions in which inhibition of smooth muscle cell proliferation is desirable, such as in arterial disease, restenosis and asthma.

Ab1 and related antibodies also are useful to enhance the immune response to macrophage-mediated infections such as those caused by Leishmania spp., Trypanosorna cruzi, Mycobacterium tuberculosis and Mycobacterium leprae, as well as the protozoan Toxoplasma gondii, the fungi Histoplasma capsulatum, Candida albicans, Candida parapsilosis, and Cryptococcus neoformans. They are also useful to reduce immunosuppression caused, for example, by tumors, AIDS or granulomatous diseases.

Ab1 and related antibodies also are useful for the prevention and/or treatment of ophthalmological conditions such as glaucoma and scarring after trabeculectomy.

B. Oncological Diseased Conditions

TGF-β regulates several biological processes, including cell proliferation, epithelial-mesenchymal transition (EMT), matrix remodeling, angiogenesis, and immune functions. Each of these processes contributes to tumor progression. The widespread detrimental role of TGF-β in cancer patients across indications also is suggested by its elevation within the tumor microenvironment as well as systemically. See, e.g., Kadam et al., Mo Biomark Diagn. (2013) 4(3):1-8. Studies have shown that in the malignant state, TGF-β can induce EMT and the resulting mesenchymal phenotype leads to increased cellular migration and invasion.

Compositions comprising Ab1 and related antibodies are useful in the treatment of hyperproliferative diseases, such as cancers including but not limited to skin cancer (e.g., melanoma, including unresectable or metastatic melanoma, cutaneous squamous cell carcinoma, and keratoacanthoma), lung cancer (e.g., non-small cell lung cancer), esophageal cancer, stomach cancer, colorectal cancer, pancreatic cancer, liver cancer (e.g., hepatocellular carcinoma), primary peritoneal cancer, bladder cancer, renal cancer or kidney cancer (e.g., renal cell carcinoma), urothelial carcinoma, breast cancer, ovarian cancer, fallopian cancer, cervical cancer, uterine cancer, prostate cancer, testicular cancer, head and neck cancer (e.g., head and neck squamous cell carcinoma), brain cancer, glioblastoma, glioma, mesothelioma, leukemia, and lymphoma.

In some embodiments, compositions Ab1 and related antibodies are useful in treating cancers in patients for whom a prior therapy based on an anti-PD-1, anti-PD-L1 or anti-PD-L2 therapeutic agent has failed or is expected to fail, i.e., patients who are or expected to be non-responders to an anti-PD-1, anti-PD-L1, or anti-PD-L2 therapy. In some embodiments, Ab1 and related antibodies are useful in the treatment of cancers in patients who have relapsed from a prior anti-PD-1, anti-PD-L1, or anti-PD-L2 therapy. As used herein, the term “expected” means that a skilled person in the medical art may anticipate, without administering a therapy, whether a patient will be a responder or a non-responder and whether the therapy will fail or will not be effective, based on his/her general medical knowledge and the specific conditions of the patient.

In some embodiments, the cancers are mesenchymal subtypes of solid tumors, including, without limitation, mesenchymal colorectal cancer, mesenchymal ovarian cancer, mesenchymal lung cancer, mesenchymal head cancer and mesenchymal neck cancer. Epithelial mesenchymal transition (EMT) promotes cellular migration and invasive properties by down regulating epithelium cell gene and enhancing mesenchymal gene expression. EMT is a hall mark of tumor progression and invasion. Up to a quarter of colorectal and ovarian cancers are mesenchymal. Thus, by inhibiting TGF-β and its induction of EMT, Ab1 or a related antibody can be used to treat mesenchymal solid tumors. Mesenchymal subtypes of solid tumors can be identified by a number of genetic markers and pathological tests. Markers include ACTA2, VIM, MGP, ZEB2, and ZWINT, which can be detected by qRT-PCR or immunohistochemistry. Such markers may be used to select patients for anti-TGFβ monotherapy or combination therapy of the invention.

In some embodiments, Ab1 and related antibodies are useful in treating patients with advanced solid tumors.

Compositions comprising Ab1 and related antibodies can also be used in the treatment of hematopoietic disorders or malignancies such as multiple myeloma, myelodysplastic syndrome (MDS), Hodgkin lymphoma, non-Hodgkin lymphoma, and leukemia, as well as various sarcomas such as Kaposi's Sarcoma.

Compositions comprising Ab1 and related antibodies can also be useful to inhibit cyclosporine-mediated malignancy or cancer progression (e.g., metastases).

It will of course be appreciated that in the context of cancer therapy, “treatment” includes any medical intervention resulting in the slowing of cancer growth, delay in cancer progression or recurrence, or reduction in cancer metastases, as well as partial remission of the cancer in order to prolong life expectancy of a patient.

C. Combination Therapy in Oncology

It has been observed that the level of cytotoxic T cell infiltration in cancer correlates with a favorable clinical outcome (Fridman et al., Nat Rev Cancer (2012) 12(4):298-306; and Galon et al., Immunity (2013) 39(1):11-26). In addition, T helper cells that assist cytotoxic T cells (CD4+TH1) and the cytokines they produce (e.g., IFN-γ) often correlate with positive patient outcomes as well. In contrast, the presence of Treg cells has been shown to correlate with a poor patient prognosis (Fridman, supra).

TGF-β suppresses almost all aspects of the anti-tumor immune response. The cytokine promotes iTreg differentiation and reduces cytotoxic (CD8+) cell proliferation and infiltration. Inhibition of TGFβ by Ab1 or a related antibody will alleviate the immunosuppressive tumor microenvironment, as described above, to bring positive outcomes to cancer patients.

Further, the inventors have discovered that by alleviating the immunosuppressive tumor microenvironment, Ab1 and related antibodies can allow checkpoint modulators, such as anti-PD-1 antibody, to better induce immune responses. As a result, more patients can benefit from immunotherapy such as anti-PD-1, anti-PD-L1, or anti-PD-L2 treatment.

With or without therapeutic agents targeting the immune checkpoint molecules, Ab1 and related antibodies can also be used in conjunction with other cancer therapies such as chemotherapy (e.g., platinum- or taxoid-based therapy), radiation therapy, and therapies that target cancer antigens or oncogenic drivers.

Cancers that can be treated by a combination involving Ab1 or a related antibody and an immune checkpoint inhibitor such as an anti-PD-1 antibody include the cancers listed in the above subsection.

In some embodiments, the cancers are refractory to a prior anti-PD-1, anti-PD-L1, or anti-PD-L2 therapy, such as advanced or metastatic melanoma, non-small cell lung cancer, renal cell carcinoma, head and neck squamous cell carcinoma, and Hodgkin Lymphoma. Refractory patients are patients whose disease progresses as confirmed, e.g., radiologically within 12 weeks of commencing treatment without any evidence of a response.

In some embodiments, Ab1 or a related antibody can be used in conjunction with another cancer therapy such as anti-PD-1 therapy to treat mesenchymal cancers such as colorectal cancer, non-small cell lung cancer, ovarian cancer, bladder cancer, head and neck squamous cell carcinoma, renal cell carcinoma, hepatocellular carcinoma, and cutaneous squamous cell carcinoma. See also discussions above.

Examples of anti-PD-1 antibodies are nivolumab, pembrolizumab, pidilizumab, MEDI0608 (formerly AMP-514; see, e.g., WO 2012/145493 and U.S. Pat. No. 9,205,148), PDR001 (see, e.g., WO 2015/112900), PF-06801591 (see, e.g., WO 2016/092419) and BGB-A317 (see, e.g., WO 2015/035606). In some embodiments, the anti-PD-1 antibodies include those disclosed in WO 2015/112800 (such as those referred to as H1M7789N, H1M7799N, H1M7800N, H2M7780N, H2M7788N, H2M7790N, H2M7791N, H2M7794N, H2M7795N, H2M7796N, H2M7798N, H4H9019P, H4×H9034P2, H4×H9035P2, H4×H9037P2, H4×H9045P2, H4×H9048P2, H4H9057P2, H4H9068P2, H4×H9119P2, H4×H9120P2, H4×H9128P2, H4×H9135P2, H4×H9145P2, H4×H8992P, H4×H8999P and H4×H9008P in Table 1 of the PCT publication, and those referred to as H4H7798N, H4H7795N2, H4H9008P and H4H9048P2 in Table 3 of the PCT publication). The disclosure of WO 2015/112800 is incorporated by reference herein in its entirety.

For example, the antibodies disclosed in WO 2015/112800 and related antibodies, including antibodies and antigen-binding fragments having the CDRs, VH and VL sequences, or heavy and light chain sequences disclosed in that PCT publication, as well as antibodies and antigen-binding fragments binding to the same PD-1 epitope as the antibodies disclosed in that PCT publication, can be used in conjunction with Ab1 or a related antibody of the present disclosure to treat cancer. In related embodiments, a useful anti-PD-1 antibody may comprise the heavy and light chain amino acid sequences shown below as SEQ ID NOs:9 and 10, respectively; the VH and VL sequences in SEQ ID NOs:9 and 10 (shown in italics); or one or more (e.g., all six) CDRs in SEQ ID NOs:9 and 10 (shown in boxes).

(SEQ ID NO: 9)

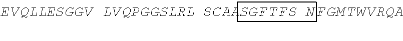

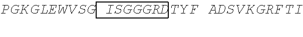

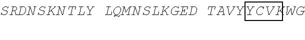

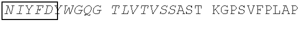

CSRSTSESTA ALGCLVKDYF PEPVTVSWNS
GALTSGVHTF PAVLQSSGLY SLSSVVTVPS
SSLGTKTYTC NVDHKPSNTK VDKRVESKYG
PPCPPCPAPE FLGGPSVFLF PPKPKDTLMI
SRTPEVTCVV VDVSQEDPEV QFNWYVDGVE
VHNAKTKPRE EQFNSTYRVV SVLTVLHQDW
LNGKEYKCKV SNKGLPSSIE KTISKAKGQP
REPQVYTLPP SQEEMTKNQV SLTCLVKGFY
PSDIAVEWES NGQPENNYKT TPPVLDSDGS
FFLYSRLTVD KSRWQEGNVF SCSVMHEALH
NHYTQKSLSL SLGK
(SEQ ID NO: 10)

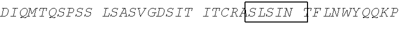

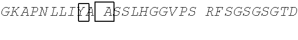

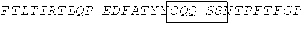

GTVVDFRRTV AAPSVFIFPP SDEQLKSGTA
SVVCLLNNFY PREAKVQWKV DNALQSGNSQ
ESVTEQDSKD STYSLSSTLT LSKADYEKHK
VYACEVTHQG LSSPVTKSFN RGEC

In other related embodiments, a useful anti-PD-1 antibody may comprise the heavy and light chain amino acid sequences shown below as SEQ ID NOs:11 and 12, respectively; the VH and VL sequences in SEQ ID NOs:11 and 12 (shown in italics); or one or more (e.g., all six) CDRs in SEQ ID NOs:11 and 12 (shown in boxes). In related embodiments, a useful anti-PD-1 antibody may comprise the heavy and light chain amino acid sequences shown below as SEQ ID NOs:11 and 12, respectively; the VH and VL sequences in SEQ ID NOs:11 and 12 (shown in italics); or one or more (e.g., all six) CDRs in SEQ ID NOs:9 and 10 (shown in boxes).

(SEQ ID NO: 11)
EVQLVESGGG LVQPGGSLRL SCAASGFTFS

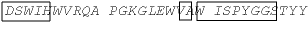

ADSVKGRFTI SADTSKNTAY LQMNSLRAED

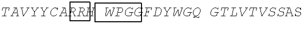

TKGPSVFPLA PSSKSTSGGT AALGCLVKDY
FPEPVTVSWN SGALTSGVHT FPAVLQSSGL
YSLSSVVTVP SSSLGTQTYI CNVNHKPSNT
KVDKKVEPKS CDKTHTCPPC PAPELLGGPS
VFLFPPKPKD TLMISRTPEV TCVVVDVSHE
DPEVKFNWYV DGVEVHNAKT KPREEQYAST
YRVVSVLTVL HQDWLNGKEY KCKVSNKALP
APIEKTISKA KGQPREPQVY TLPPSREEMT
KNQVSLTCLV KGFYPSDIAV EWESNGQPEN
NYKTTPPVLD SDGSFFLYSK LTVDKSRWQQ
GNVFSCSVMH EALHNHYTQK SLSLSPGK
(SEQ ID NO: 12)

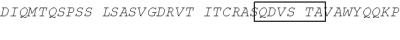

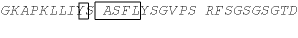

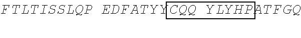

GTKVEIKRTV AAPSVFIFPP SDEQLKSGTA
SVVCLLNNFY PREAKVQWKV DNALQSGNSQ
ESVTEQDSKD STYSLSSTLT LSKADYEKHK
VYACEVTHQG LSSPVTKSFN RGEC

In some embodiments, the antibodies of the present disclosure, such as the anti-PD-1 antibodies, do not have the C-terminal lysine in the heavy chain. The C-terminal lysine may be removed during manufacture or by recombinant technology (i.e., the coding sequence of the heavy chain does not include a codon for the C-terminal terminal lysine). Thus, contemplated within the invention also are antibodies comprising the heavy chain amino acid sequence of SEQ ID NO: 3 without the C-terminal lysine.

D. Biomarkers of Treatment Efficacy

Efficacy of Ab1 and related antibodies can be determined by biomarkers or target occupancy. For example, in tumor tissues, target occupancy can be assayed by evaluating levels of active TGFβ in biopsies using a Meso Scale Discovery (MSD) assay. In the blood, target engagement can be assayed by evaluating the effect of decreased circulating TGFβ on peripheral blood mononuclear cells such as lymphocytes (T cells, B cells, NK cells) and monocytes. For example, increased proliferation of circulating CD8+ T cells can be evaluated using CD45⁺RO⁺CCR7⁺CD28⁺Ki67⁺ as markers in flow cytometry. Activation of circulating NK cells can be evaluated using CD3-CD56high/dim CD16⁺ or CD137⁺ as markers in flow cytometry. Additionally, Ki-67, PD-1, and ICOS can be used as PD markers associated with T cell activation.

Immune modulation upon treatment by Ab1 or a related antibody can be assayed by evaluating changes of infiltrating immune cells and immune markers by multiplex immunohistochemistry (IHC) assays using, e.g., the NeoGenomics platform. Specifically, NeoGenomic's MultiOmyx TIL Panel stains for a panel of immune markers, allowing for quantitative determination of density and localization of various immune cells. The immune markers may indicate differentiation of iTreg; infiltration and proliferation of CD8⁺ T cells; and generation of IFN γ by CD8⁺ T cells. Ab1 has been shown to inhibit CD4⁺ T cells' differentiation into iTreg (see, e.g., Example 3 in US Patent Pub. No. US2018/0244763), and to increase CD8⁺ T cell proliferation and their generation of IFN γ (as shown in a mixed lymphocyte reaction assay; data not shown). Thus, efficacy of treatment by Ab1 or a related antibody can be indicated by inhibition of iTreg, induction of CD8⁺ T cell proliferation and infiltration to tumor or other diseased tissues, increased IFN γ production, and/or an increased ratio of CD8⁺ T cells to Treg cells. Immune modulation upon treatment by Ab1 or a related antibody also can be assayed in peripheral blood by methylation-PCR based quantitative immune cell counting of CD8⁺ T cells, Treg cells, NK cells, and other immune cells. The treatment efficacy may manifest clinically as a delay or reversal in disease progression such as tumor progression.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of neurology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### EXAMPLES

The Examples below describe studies that evaluated various formulations for Ab1 in order to arrive at formulations with the best biological activity and long-term stability. We evaluated the effects of buffer identity and pH on the physical and chemical stability of Ab1 liquid formulations during refrigerated storage, accelerated storage, and stressed storage conditions. Acetate and histidine buffer systems were chosen for this study with and without the addition of sodium chloride.

We assessed the optimal concentrations of polysorbate 80 (PS80) needed to stabilize Ab1 liquid formulations at various storage temperatures, freeze-thaw cycling, and agitation-induced stress.

We also evaluated the physical stability of Ab1 drug product (DP) after dilution and incubation for up to 48 hours at room temperature in intravenous (IV) infusion bags. The DP was diluted to 0.5 mg/ml and 1.0 mg/ml. Both concentrations were evaluated in the following bag combinations: saline in polyvinyl chloride (PVC) bags, saline in polyolefin (PO) bags, dextrose in PVC bags, and dextrose in PO bags. The optimal PS80 concentration in the liquid DP was also examined for its ability to protect Ab1 after dilution.

We further examined the impact of transition metals on the chemical and physical stability of the protein. Transition metals are sometimes leached into the drug substance (DS) during manufacturing. EDTA and DTPA were evaluated for their abilities to chelate and protect the protein during a set of worst-case experiments.

To ensure that the proposed target formulation matrix is sufficient to stabilize high-concentration DS and lower-concentration DP solutions, we assessed the solutions' stability during freeze-thaw cycles and under frozen and liquid storage conditions, and across concentration ranges for all excipients, including the API.

The materials and methods for the experiments are as follows.

Drug Substance

High-concentration Ab1 drug substance (DS) was prepared using ultrafiltration/diafiltration methods (UFDF). The DS concentration was generally prepared at up to 180 mg/ml. Results from the UFDF simulator was used for concentration correction of buffer salts that may be accumulated or depleted during UF process steps due to the Donnan effect. After formulating drug product (DP) samples to the target protein and excipient concentrations, formulations were then purified with 0.22 μm filters under laminar flow before aseptic filling.

Inspection for Visible Particles

Visible particles were analyzed under a visual inspection unit. DP vials were cleaned with lens paper before inspection to remove dust and fingerprints from the outer surface.

pH

The pH of buffers and formulated mAb solutions was measured using a Thermo-Scientific™ pH probe and meter. The results were considered comparable when the difference between repeated measurements was within 0.1 pH units.

Osmolality

Osmolality measurements were performed on 20 μL samples (n=2 or 3) using a freezing point depression osmometer (Advanced Instruments, OsmoPRO). Osmolality standards were run before and after sample analysis to ensure measurement accuracy.

Total Protein Concentration

Total protein concentration was determined by measuring the ultraviolet (UV) absorbance at 280 nm using the Variable Pathlength Technology by a SoloVPE system from C technologies. Measurements were performed on 20 μL of sample (n=2 or 3). Total protein concentration was also determined by measuring the UV absorbance at 280 nm on microfluidic chips on a Big Lunatic system from Unchained Labs. Measurements were performed in duplicate on 2 to 5 μL of sample.

DSC for Conformational and Thermal Stability

Differential scanning calorimetry (DSC) was performed on a Malvern Microcal calorimeter by thermal ramping from 15° C. to 105° C. at a heating rate of 0.5° C./min. The protein solutions were measured at 1 mg/ml protein concentration. OriginPro software was used for analysis and thermal unfolding temperature (Tₘ) determination.

Solution Turbidity and Optical Density

Sample turbidity was quantified by measuring optical density (OD) from 340 nm to 360 on a SpectraMax® i3 Microplate Reader from Molecular Devices. For each sample, 200 μL was loaded onto a UV-Vis transparent 96 well plate. The OD was determined as the average of absorbance values at 340 nm, 345 nm, 350 nm, 355 nm, and 360 nm.

Size-Exclusion HPLC for High Molecular Weight Species

Analysis of protein aggregates (high molecular weight species or HMWS) was performed by size-exclusion chromatography (SEC). Samples were run on a 1260 series HPLC (Agilent, Santa Clara, Calif.) equipped with a TSK-GEL® G3000SWXL (Tosoh Bioscience, Tokyo, Japan) analytical column and a matching guard column. The mobile phase used was 40 mM phosphate and 150 mM sodium chloride, pH 7.2 with a flowrate of 0.5 mL/min for 30 minutes. Three injections were performed for each sample. Detection was carried out by UV absorbance at 280 nm and the chromatographic peaks were integrated to determine the relative percentage of each eluted species.

Micro-Flow Imaging (MFI) for Subvisible Particles

Subvisible particles were analyzed using a Protein Simple MFI™ Model DPA-4200. The system was extensively flushed with 0.22 μm filtered and degassed MilliQ® water before measuring 2, 10, and 25 μm standards. Samples (n=1 or 2) were run using a 1 mL method at a flow rate of 0.17 mL/min. As sample flowed through the flow cell, it was illuminated by a light source and a camera rapidly captured images as the sample passed through the flow cell. The particles were identified by the MFI™ software, which then calculated the size, transparency, and morphology of every individual particle.

High Accuracy Liquid Particle Counter for Subvisible Particles

Subvisible particles were also measured by light obscuration on a Hach® high accuracy (HIAC) liquid particle counter Model 9703+. The system was flushed with 0.22 μm filtered and degassed MilliQ® water until particle counts were below 20 particles/mL. 2 μm, 10 μm, and 25 μm standards were measured to ensure accurate particle counts followed by extensive washing to remove any background. Using a 1 mL protocol, samples were measured with five separate injections of 0.2 mL. The first sample measurement was disregarded and the following four were averaged.

Capillary Isoelectric Focusing for Charge Variants

The protein charge heterogeneity was measured by capillary isoelectric focusing (cIEF) using an iCE3 instrument from ProteinSimple by UV absorbance at 280 nm. The samples (1 mL) and the standard solutions were diluted to 2.5 mg/ml in water. Onboard mixing was used to mix samples and Master Mix before analyses. The isoelectric focusing of the samples included a pre-focusing time of 1 minute at 1500 V followed by a focusing over 10 minutes at 3000 V. The detection covered 5 exposures and the sample loading lasted 55 seconds for each formulation. The results were considered comparable when the difference was equal or less than 10%.

PTMs Quantification by LC-MS

Protein samples were diluted to 2 mg/ml, vortexed, and 40 μg of protein was used for automated digestion. The digest buffer was 25 mM Tris, pH 8.5. For each sample, 15 μL of the digest (containing about 5 μg of protein) was injected onto a C18 column for LC-MS analysis. The samples were analyzed using DDA top 8 LC-MS/MS method on Q Exactive™.

The LC-MS/MS data acquired on Q Exactive™ were processed by BioPharma Finder™ 3.0 on server WLSD58 for identification and relative quantitation of modifications including Met/Trp oxidation, deamidation, Asp isomerization and HC C-terminal modification. For low level of modifications that could not generate good MS/MS spectra for identification by BioPharma Finder™, MS only peptide mapping was used for peptide assignment. All data were then processed using Progenesis to provide peptide abundance after retention time alignment and peak picking. Manual adjustment of the peak picking was required for some deamidated and isomerized peptides when Progenesis was unable to correctly perform this task.

Potency

When TGF-β is incubated with mink lung cells, it inhibits cell proliferation. Ab1 is an anti-TGFβ antibody that when bound to TGF-β, inhibits TGF-β from binding to the TGF-β cell surface receptors, thus allowing cell proliferation. In the Ab1 potency assay, varying levels of Ab1 were incubated with TGF-β2 and then added to mink lung cells.

The cells were incubated with Ab1 and TGF-β2 for three days and then PrestoBlue™ reagent was added. PrestoBlue™ contains a cell-permeable, non-fluorescent compound, resazurin, which is metabolized and reduced by living cells, resulting in the production of the fluorescent product, resorufin. Therefore, cell proliferation is directly correlated with the intensity of fluorescent signal. Five hours after addition of PrestoBlue™, the fluorescence was measured using a plate reader. Bioassay software, Log₁₀ transformed and fit to a four-parameter model. After the reference and sample curves were determined to be suitable, the curves were constrained and the final result of the assay was determined as a ratio of the EC₅₀ of the reference divided by the EC₅₀ of the test sample and reported as percent relative potency (% RP).

### Example 1: Buffer and pH Screen

This Example describes experiments in which various buffers and pH conditions were screened to identify suitable formulations for Ab1. Because liquid drug products are more convenient for preparation and administration in both clinical and at-home settings (as compared to lyophilized drug products), various liquid aqueous buffers were tested. Table 1 represents formulation conditions and sample codes used in this study.

TABLE 1
Buffer Conditions

| Reference | Condition | Ab1 (mg/ml) |
|---|---|---|
| Ace_4.7 | pH 4.7, 20 mM acetate | 80 |
| Ace_5.0 | pH 5.0, 20 mM acetate | 80 |
| Ace_5.3 | pH 5.3, 20 mM acetate | 80 |
| Ace_5.5 | pH 5.5, 20 mM acetate | 80 |
| Ace-NaCl_5.5 | pH 5.5, 20 mM acetate, 25 mM NaCl | 80 |
| Hist-NaCl_5.5 | pH 5.5, 10 mM histidine, 25 mM NaCl | 80 |
| Hist_5.5 | pH 5.5, 10 mM histidine | 80 |
| Hist_6.0 | pH 6.0, 10 mM histidine | 80 |
| Hist_6.5 | pH 6.5, 10 mM histidine | 80 |

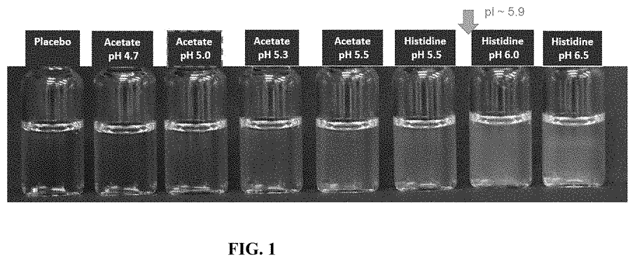

Opalescence is a visual manifestation of attractive protein-protein interactions. It was visually observed that Ab1 formulations exhibited significant pH-dependent opalescence (FIG. 1). Solutions were mostly clear and transparent below pH 5.3, but at pH>5.3, the opalescence increased with increasing pH. Acetate formulations generally had lower opalescence than histidine formulations (FIG. 1). These visual appearance images suggest an optimal formulation pH range of 4.7-5.0 using acetate as the buffering species.

After up to 4 weeks of storage at 5° C. and 25° C., the solutions in Table 1 showed no significant change in high molecular weight species (HMWS) %. There was, however, about a 0.5% increase in HMWS % for all formulations after 4 weeks of storage at 40° C. This change was not particularly significant given the stressed conditions. Furthermore, the addition of sodium chloride to the formulations did not have a significant impact on the extent of protein aggregation and HMWS %.

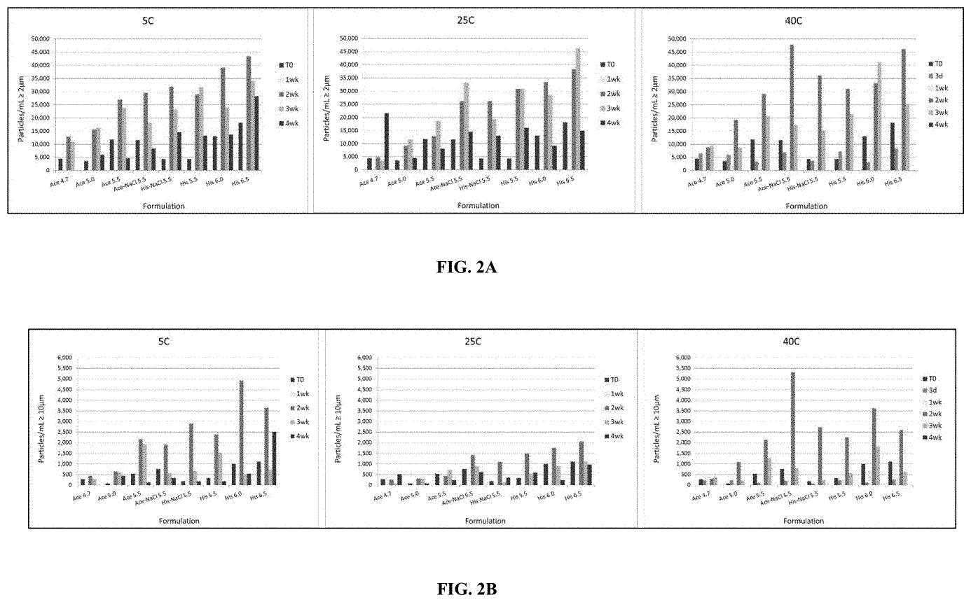

With regard to subvisible particles, HIAC particulate counts show that subvisible particle growth was sensitive to the identity of the buffer species and solution pH (FIGS. 2A-2C). Acetate formulations buffered at pH 4.7 and 5.0 resulted in the smallest numbers of particles generated over time compared to any other pH of acetate formulations, and to all histidine formulations studied. There was not, however, a discernable or clear effect of temperature on particle growth for any condition.

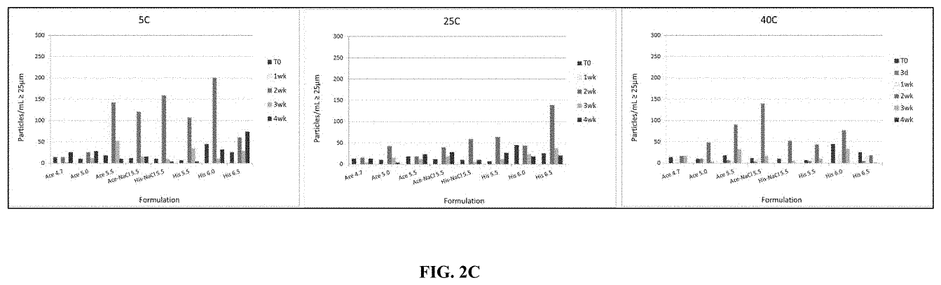

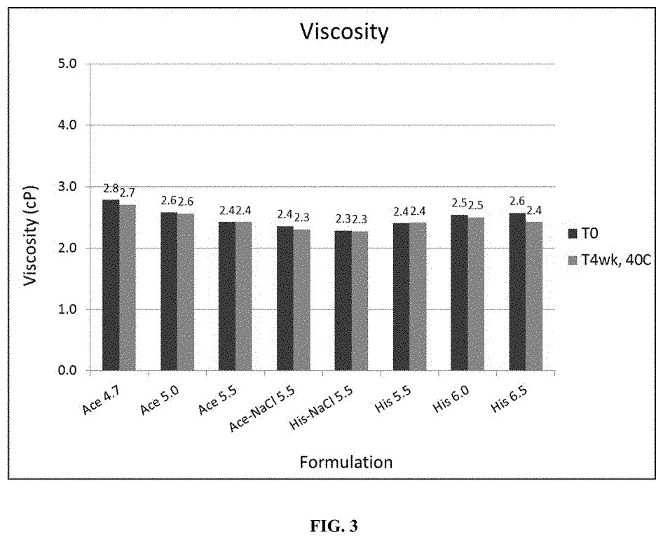

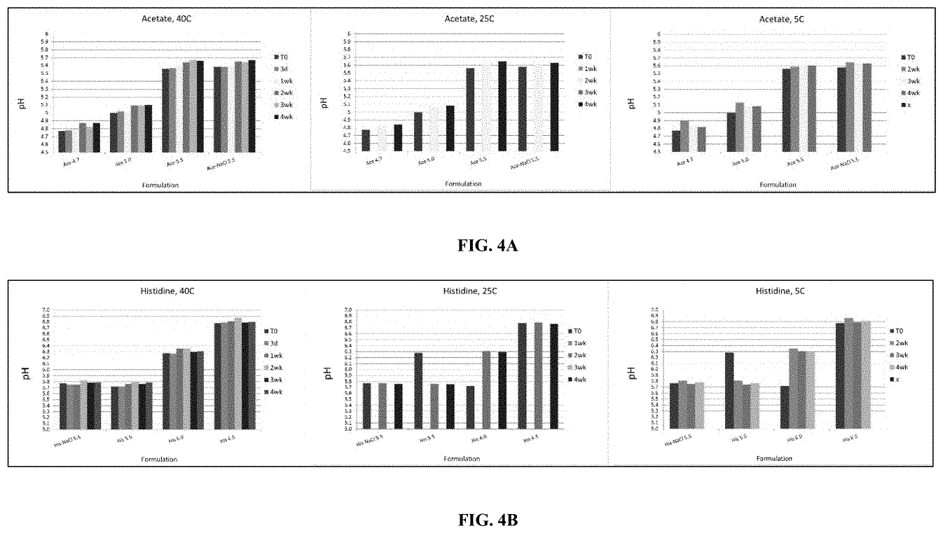

Acetate and histidine formulations had comparable viscosities at T₀ and after 4 weeks at 40° C. for any given pH, with or without the addition of sodium chloride (FIG. 3). The measured viscosity values were all within 2-3 cP, which is well below any limits that may present challenges during DP administration to patients, or during manufacturing processes. There was also no significant change in viscosity values, indicating that the tested formulations did not undergo significant chemical degradation. Over the four-week period, there also was no significant change in the pH value of any formulation after storage at the three temperatures (FIGS. 4A and 4B). These results suggest that acetate and histidine did not undergo any significant chemical degradation and maintained their ability to buffer even after up to 4 weeks of stressed storage at 40° C.

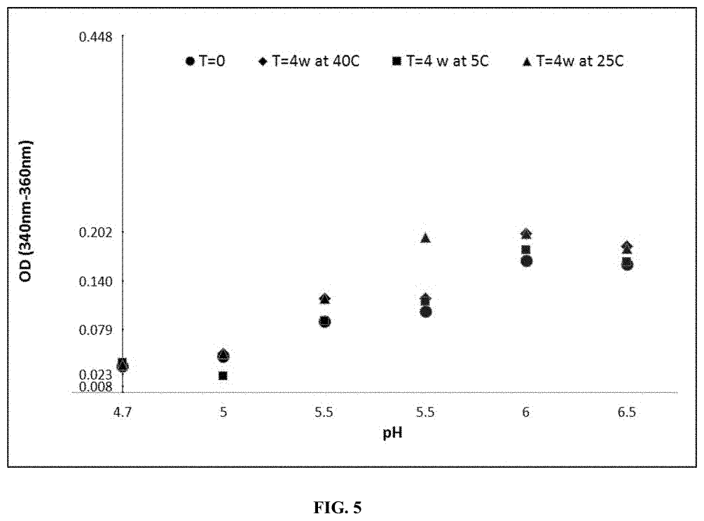

Plate UV measurements were made to track any changes in turbidity and opalescence that may have occurred over time (FIG. 5). The same pH dependent opalescence observed during visual inspection previously at T₀ was detected spectrophotometrically. The measured OD values increased with pH, with histidine formulations being more turbid than acetate formulations. There was a slight increase in OD for most of the formulations after 4 weeks of storage at 25° C. and 40° C. The increases in OD over time were due to the formation of subvisible particles. The results confirm that a pH around 4.7-5.0 led to the least amount of subvisible particles.

The osmolality values of buffer only and buffered protein solutions were compared (Table 2). Acetate formulations were slightly higher in osmolality than histidine. The addition of protein increased the osmolality of all formulations. The osmolality values are all reasonable given that the DP is administered only after dilution into an IV infusion bag.

TABLE 2
Osmolarity Values of Buffer Only and Buffered Ab1 Solutions

Osmolality (mOsmo/kg)

| Sample | Buffer Only (dupl.) | 80 mg/ml Ab1 |
|---|---|---|
| Ace_4.7 | 24.5 | 92 |
| Ace_5.0 | 28 | 40 |
| Ace_5.5 | 32 | 46 |
| Ace-NaCl 5.5 | 79.5 | 101 |
| His-NaCl 5.5 | 71.5 | 74 |
| Hist_5.5 | 13.5 | 26 |
| Hist_6.0 | 11.5 | 21 |
| Hist_6.5 | 9 | 15 |

The chemical stability of Ab1 was similar for acetate and histidine formulations as seen when comparing quantities of acidic isoforms and monomer percentages. There was a slight increase in the relative amount of acidic isoform generated as solution pH approached 6.0 and 6.5 for histidine formulations after 4 weeks at 40° C.

All formulations had the same relative potency of about 1.0, suggesting that they are all acceptable. These potency results are in line with the previous aggregation and HMWS % results, as all HMWS % values were low (<2%) and therefore potency is not expected to change considerably.

### Example 2: Surfactant Screen

This Example describes experiments evaluating the addition of surfactant polysorbate 80 (PS80) to Ab1 formulations. The formulation conditions and sample codes used in these experiments are shown below in Table 3.

TABLE 3
Formulations with Polysorbate 80

| Formulation | PS80 Source | Reference | PS80 | Ab1 | Buffer | pH |
|---|---|---|---|---|---|---|
| 1 | A | PS80_CSR_0% | 0% | 150 | 20 mM acetate + | 5.0 |
| 2 | | PS80_CSR_0.025% | 0.025% | mg/ml | 8% sucrose | |
| 3 | | PS80_CSR_0.05% | 0.05% | | | |
| 4 | | PS80_CSR_0.1% | 0.1% | | | |
| 5 | B | PS80_ChP_0.05% | 0.05% | 150 | 20 mM acetate + | 5.0 |
| 6 | | PS80_ChP_0.1% | 0.1% | mg/ml | 8% sucrose | |

Plate turbidity and optical density (OD; at 340-360 nm) values for the formulations were evaluated for formulations stored for one week at 5° C. or 40° C., or after 48 hours of vigorous agitation. The data show that the OD values did not change after 1 week of storage at either 5° C. or 40° C., or after 48 hours of agitation, for formulations containing PS80. There were also no differences observed with varying concentrations 0.025%, 0.05% and 0.1% of PS80 using two different commercial sources of PS80 (A and B). The OD values did slightly decrease, however, for formulations without added surfactant over time at both 5° C. and 40° C. storage. There was also slightly higher turbidity observed for the surfactant-free formulations. These observations suggest that PS80 had a solubilizing effect on the Ab1 protein.

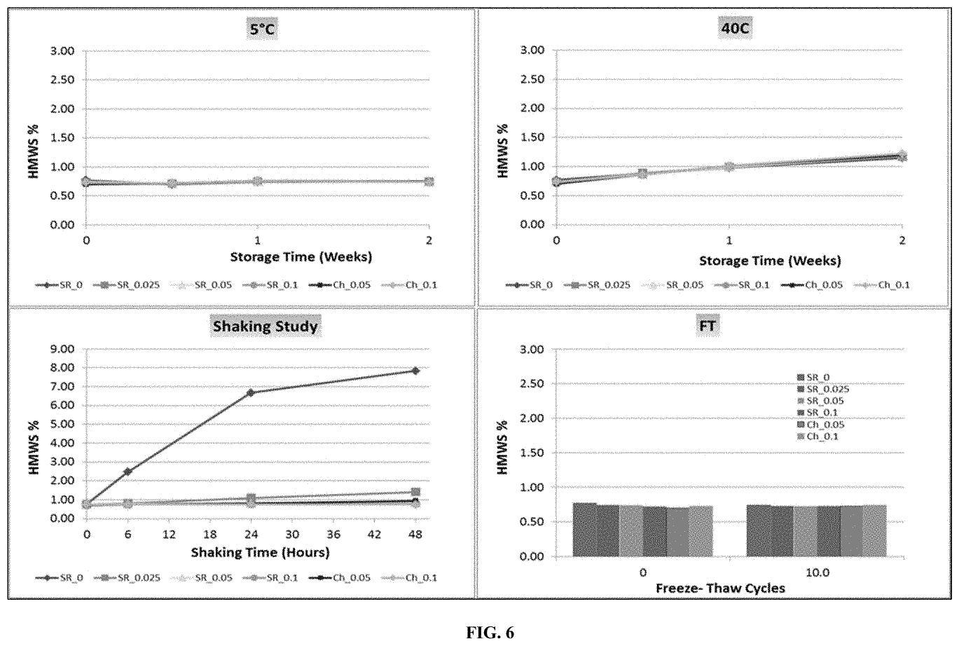

The levels of small soluble aggregates were monitored by SEC under different storage and interfacial stress conditions (FIG. 6). Storage temperature had only a marginal impact and led to 0.5% increase of aggregation after up to 2 weeks of storage; there was no noticeable effect of PS80 concentration on aggregation levels. Worst case freeze-thaw cycling between −30° C. and room temperature up to 10 times did not have a negative impact on protein stability, either (FIG. 6, bottom-right panel).

Rigorous agitation or shaking, on the other hand, had a great impact on aggregation (FIG. 6, bottom-left panel). Formulations without added surfactant generated up to 8% HMWS after 48 hours of agitation. These aggregation levels were highly sensitive to the presence and concentration of surfactant. PS80 concentrations as low as 0.025% were enough to decrease aggregation levels down to less than 2% of the aggregation levels of PS80-free formulations. Increasing the PS80 concentration even higher to 0.05 or 0.1% marginally decreased the HMWS % further. These data suggest that 0.05% could be used as the lower limit of PS80 concentration targeted to stabilize Ab1 formulations.

The above SEC data suggest that Ab1 does not have a high risk of aggregation for most of the conditions studied in this section. HIAC was therefore a critical assay needed for tracking larger soluble and insoluble aggregates filtered out by SEC which can be sized in the subvisible particle range. HIAC data show that under any given storage or interfacial stress condition, formulations that did not contain any PS80 generated substantially more subvisible particles. The data further show that PS80 had a concentration-dependent effect on reduction of the number of particles in Ab1 formulations. PS80 concentrations as low as 0.025% were enough to begin to decrease particle counts. Increasing the PS80 concentration to 0.05% and higher continuously decreased particle counts.

The pH values of the formulations were measured at T₀ and after 2 weeks of storage. There were no significant changes in pH for any formulations at any time points or temperatures. There was also no concentration-specific PS80 influence on formulation pH. The concentrations of PS80 did not induce any changes on the ability of the acetate buffer to maintain pH of 150 mg/ml Ab1 formulations.

For all the studies in this Example, there did not appear to be an effect of PS80 source on formulation stability for any of the test conditions.

### Example 3: IV Bag Dilution Study

This Example describes experiments testing the stability of Ab1 formulations in different IV bags. The formulation conditions used in these experiments are shown below in Table 4.

TABLE 4
Formulations and Sample Codes for IV Bag Dilution Study
PO

| Diluted | PO_Dextrose | | PO_Saline | |
|---|---|---|---|---|
| PS80 (%) | 0.5 mg/ml Ab1 | 1.0 mg/ml Ab1 | 0.5 mg/ml Ab1 | 1.0 mg/ml Ab1 |
| 0.0010% | PO_D_0.5_a | PO_D_1.0_a | PO_S_0.5_a | PO_S_1.0_a |
| 0.0005% | PO_D_0.5_b | PO_D_1.0_b | PO_S_0.5_b | PO_S_1.0_b |
| 0.0003% | PO_D_0.5_c | PO_D_1.0_c | PO_S_0.5_c | PO_S_1.0_c |
| 0.0002% | PO_D_0.5_d | PO_D_1.0_d | PO_S_0.5_d | PO_S_1.0_d |

PVC

| Diluted | PVC_Dextrose | | PVC_Saline | |
|---|---|---|---|---|
| PS80 (%) | 0.5 mg/ml Ab1 | 1.0 mg/ml Ab1 | 0.5 mg/ml Ab1 | 1.0 mg/ml Ab1 |
| 0.0010% | PVC_D_0.5_a | PVC_D_1.0_a | PVC_S_0.5_a | PVC_S_1.0_a |
| 0.0005% | PVC_D_0.5_b | PVC_D_1.0_b | PVC_S_0.5_b | PVC_S_1.0_b |
| 0.0003% | PVC_D_0.5_c | PVC_D_1.0_c | PVC_S_0.5_c | PVC_S_1.0_c |
| 0.0002% | PVC_D_0.5_d | PVC_D_1.0_d | PVC_S_0.5_d | PVC_S_1.0_d |

Before IV infusions, the DP is diluted into IV bags. Without sufficient concentrations of surfactant in the DP, the protein molecules could possibly adsorb to the bag depending on the type of material that the IV bag is made of. As a result, the diluted DP in the IV bag could have a lower API concentration than intended for the dose administered to the patient.

We tracked the adsorption behavior and physical stability of Ab1 diluted into bags that were spiked and coated with different concentrations of PS80. PS80 was diluted by spiking to concentrations of 0.001%, 0.0005%, 0.0003%, and 0.0002% in order to determine the optimal concertation needed in the starting DP formulation. The aforementioned PS80 concentrations would correspond to 50-300 fold diluted PS80 concentrations in the starting DP. Next, 150 mg/ml DP not containing PS80 was diluted to 0.5 or 1.0 mg/ml. The effects of diluting the formulations into four different combinations of IV bag materials and dilutes was evaluated: saline in PVC bags, saline in PO bags, dextrose in PVC bags, and dextrose in PO bags.

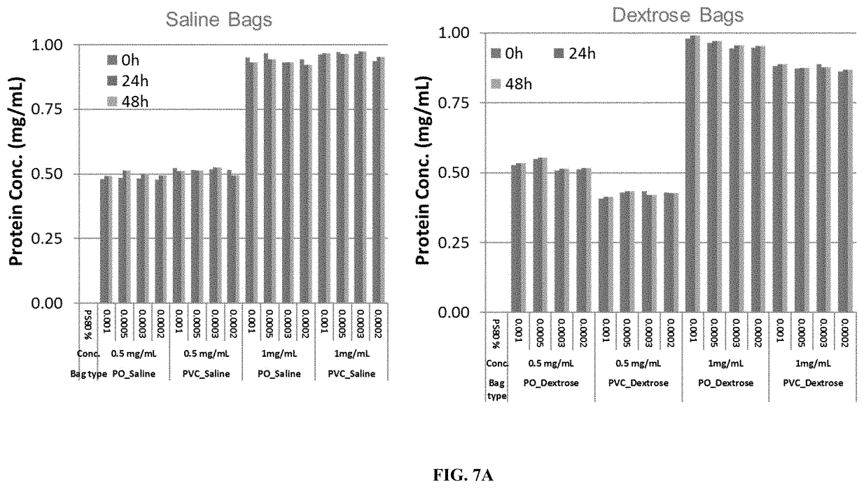

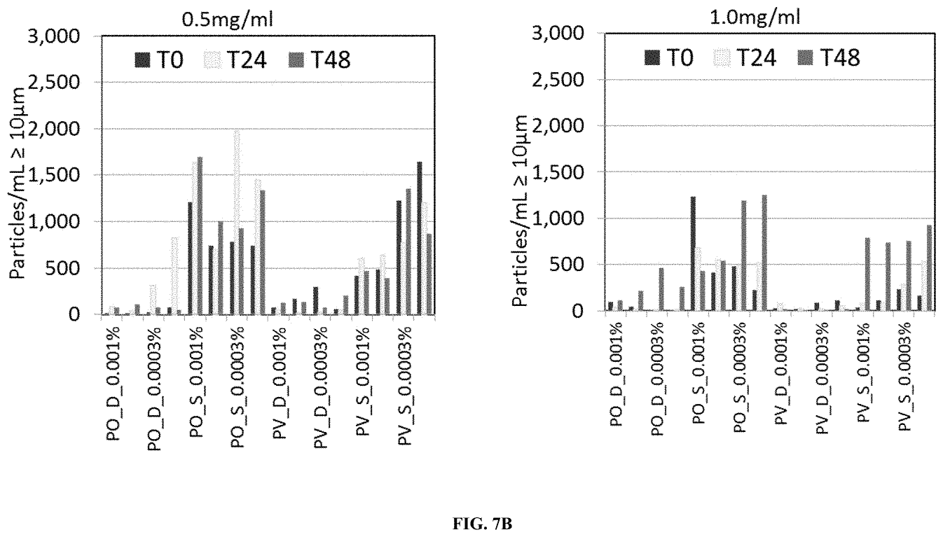

Diluted formulations were then incubated and measured after 24 and 48 hours. The data show that saline in PO and PVC bags did not significantly influence protein adsorption for any spiked surfactant concentration studied (FIG. 7A). Using dextrose as the diluent did, however, impact the adsorption slightly in a manner dependent on bag material. Specifically, PVC dextrose bag combinations caused noticeable protein adsorption as PS80 concentration decreased (FIG. 7A). The results directly impact recommendations that will be made for administering the DP.

Subvisible (>10 μm) particle generation after DP dilution was highly sensitive to the type of diluent used. Saline-based solutions generated higher levels of particles than dextrose-based solutions (FIG. 7B). This was regardless of the concentration of PS80 used in the formulation. There was not a significant difference between subvisible particle counts between 0.001% and 0.003% PS80. The material of the IV bag did not appear to have a significant impact of particulation and particle counts were comparable.

The aggregation of diluted DP samples after incubation in dextrose and saline with PO and PVC bag combinations was also evaluated. Protein diluted into saline bags generated slightly more HMWS % than that of dextrose bags. PO bags also generated more aggregates that PVC bags. There was also a protein concentration dependence on protein stability where the lower 0.5 mg/ml dilutions generally had higher aggregation levels than the 1.0 mg/ml dilutions. PS80 concentration, however, did not have a significant impact on protein stability.

### Example 4: DS and DP Stability

This Example describes studies for evaluating the long-term stability of the drug substance (DS) and drug product (DP). Table 5 shows the formulations tested, where sucrose was used as cryoprotectant, PS80 was used as a surfactant, and DTPA was used as a chelator.

TABLE 5
Formulations for Long-Term Stability Test

| | Ab1 | Buffer | | | DTPA |
|---|---|---|---|---|---|
| Ref # | (mg/ml) | (pH 5.0) | % sucrose | % PS80 | (μM) |
| D_1 | 50 | 20 mM acetate | 8 | 0.06 | 50 |
| D_2 | 100 | 20 mM acetate | 8 | 0.06 | 50 |
| D_3 | 135 | 20 mM acetate | 8 | 0.06 | 50 |
| D_4 | 150 | 20 mM acetate | 8 | 0.06 | 50 |
| D_5 | 165 | 20 mM acetate | 8 | 0.06 | 50 |

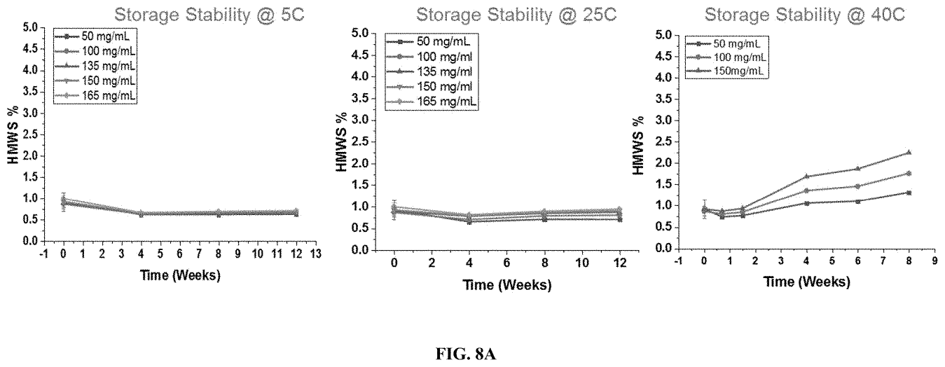

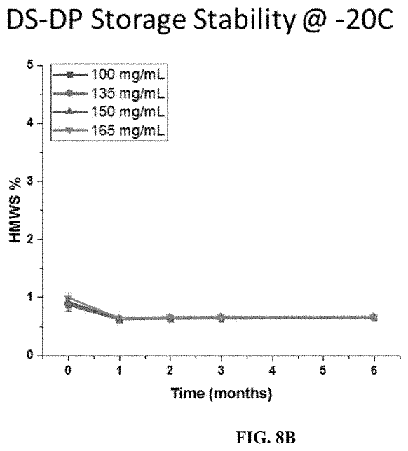

Given these data on 20 mM acetate buffers containing 50 μM DTPA, it was determined that formulations of UF/DF-purified Ab1 containing 25 mM acetate, which would provide the same pH range, and 10 μM EDTA would display the same solution behavior. To that end, we tested this preliminary proposed target formulation for DS and DP: 25 mM acetate, 8% sucrose, 0.06% PS80, 10 μM chelator (tested DTPA, but EDTA being equivalent), pH 5.0 (Table 5). The ability of this formulation matrix to stabilize Ab1 at concentrations that span a range of DS and DP concentrations was assessed on storage stability. After 3 months of storage at 5° C. or 25° C., there was not a significant change in HMWS % in any of the formulations (FIG. 8A). In the 40° C. arm, there was up to about a 0.5% increase; however, this increase is considered marginal under these types of accelerated stressed storage conditions and reflects the overall ability of the proposed formulation matrix to optimize stability and shelf life.

The Ab1 formulations were also frozen at −80° C. and then stored at −20° C. There was no significant change in HMWS % after 6 months of storage under these conditions (FIG. 8B). Low molecular weight species (LMWS) % was also tracked by SEC, and there were no signification changes in fragmentation observed under any formulation or storage condition. Further, every formulation was within USP <787> specifications for both 10 and 25 μm subvisible particles.

There also were no dramatic changes in the chemical stability of Ab1 under any condition. In sum, the proposed formulation matrix has the ability to stabilize the range of the lowest potential DP concentration of 50 mg/ml and the highest potential DS concentration of 165 mg/ml.

### Example 5: Metal Spiking Study and Chelator Compatibility

There is a low risk of transition metal contamination in the DS and DP during manufacturing of biologics. If there is contamination, the transitional metals can lead to chemical instability and aggregation in the liquid solution. This Example describes studies that evaluated the impact of metal and chelators on Ab1 formulations. Table 6 shows the formulations used to test metal spikes:

TABLE 6
Formulations for Metal Spiking Study

| | Ab1 | Buffer | % | % | DTPA | Fe | Cu |
|---|---|---|---|---|---|---|---|
| Ref # | (mg/ml) | (pH 5.0) | sucrose | PS80 | (μM) | (ppb) | (ppb) |
| M_1 | 150 | 20 mM | 8 | 0.06 | — | — | — |
| | | acetate | | | | | |
| M_2 | 150 | 20 mM | 8 | 0.06 | — | — | 500 |
| | | acetate | | | | | |
| M_3 | 150 | 20 mM | 8 | 0.06 | 50 | — | 500 |
| | | acetate | | | | | |
| M_4 | 150 | 20 mM | 8 | 0.06 | — | 500 | — |
| | | acetate | | | | | |
| M_5 | 150 | 20 mM | 8 | 0.06 | 50 | 500 | — |
| | | acetate | | | | | |

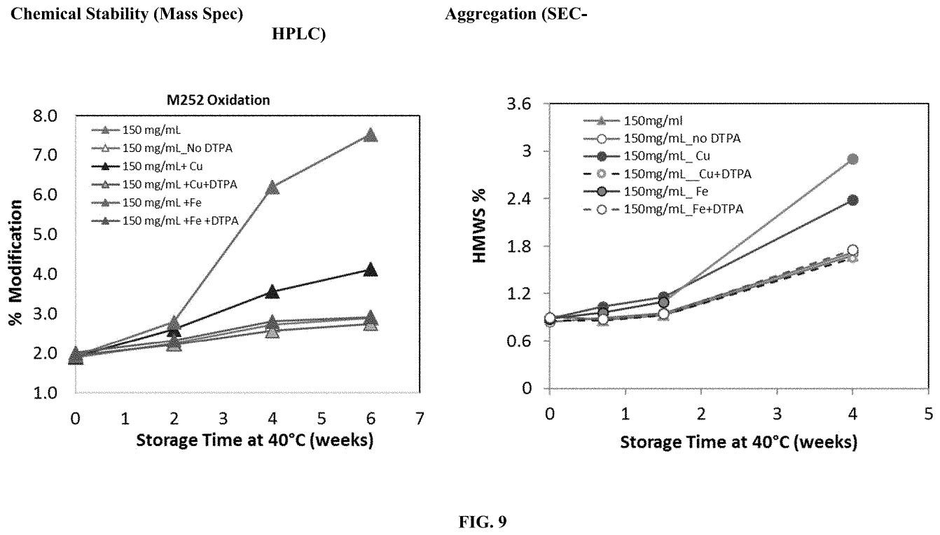

As shown in the table, Ab1 samples were spiked with iron and copper, with and without the addition of chelator DTPA. Without added chelator, there was significant M252 oxidation and aggregation in iron-spiked samples; copper-spiked samples also displayed increased oxidation and aggregation, but to a lesser extent (FIG. 9). In the presence of DTPA, however, there was a dramatic decrease in protein degradation.

We also tested another chelator, EDTA. The storage stability of Ab1 formulations were spiked with 10 μM DTPA or 10 μM EDTA and were compared. The data show that these two chelators provided similar levels of protection for the protein molecules in solution. These chelators selectively mitigate metal-induced particle formation or aggregation.

### Example 6: Formulation Robustness with Concentration Ranges for API and Excipients

This Example describes studies that evaluated the stability of Ab1 formulations across concentrations ranges of the antibody as well as the excipients. The formulations used for the studies are shown in Table 7 below.

TABLE 7
Formulations for API and Excipient Concentration Range Studies

| | | Ab1 | Acetate | % | % | DTPA |
|---|---|---|---|---|---|---|
| Ref by pH | pH | (mg/ml) | (mM) | sucrose | PS80 | (μM) |
| 4.7_F1 | 4.7 | 40 | 16 | 9.6 | 0.045 | 50 |
| 4.7_F2 | 4.7 | 40 | 24 | 6.4 | 0.075 | 50 |
| 4.7_F3 | 4.7 | 40 | 24 | 9.6 | 0.060 | 40 |
| 4.7_F4 | 4.7 | 57.5 | 16 | 6.4 | 0.045 | 40 |
| 4.7_F5 | 4.7 | 75 | 16 | 8.0 | 0.075 | 50 |
| 4.7_F6 | 4.7 | 75 | 20 | 9.6 | 0.075 | 40 |
| 4.7_F7 | 4.7 | 75 | 24 | 6.4 | 0.045 | 45 |
| 5.0_F1 | 5.0 | 40 | 16 | 6.4 | 0.075 | 40 |
| 5.0_F2 | 5.0 | 57.5 | 20 | 8.0 | 0.060 | 45 |
| 5.0_F3 | 5.0 | 75 | 24 | 9.6 | 0.045 | 50 |
| 5.3_F1 | 5.3 | 40 | 16 | 9.6 | 0.075 | 45 |
| 5.3_F2 | 5.3 | 40 | 20 | 6.4 | 0.045 | 50 |
| 5.3_F3 | 5.3 | 40 | 24 | 8.0 | 0.045 | 40 |
| 5.3_F4 | 5.3 | 57.5 | 24 | 9.6 | 0.075 | 50 |
| 5.3_F5 | 5.3 | 75 | 16 | 6.4 | 0.060 | 50 |
| 5.3_F6 | 5.3 | 75 | 16 | 9.6 | 0.045 | 40 |
| 5.3_F7 | 5.3 | 75 | 24 | 6.4 | 0.075 | 40 |

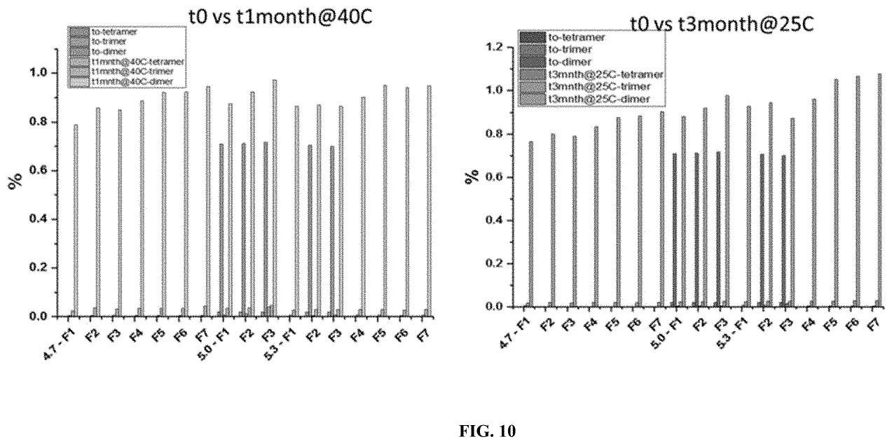

We evaluated aggregation by analyzing HMWS as subspecies of dimer, trimer, and tetramer. This allowed for the ability to track the influence of variations in the formulation components directly to the details of aggregation. pH 4.7 and pH 5.3 were the most stable and did not generate large quantities of HMWS; furthermore, the dominate HMW species was dimer (FIG. 10). pH 5.0 formulations generated roughly 0.6% more total aggregate; the dimer levels were comparable to pH 4.7 and 5.3 with the 0.6% representing primarily trimer species. Nonetheless, the total HMWS % level was acceptable.

There was no dramatic change in the number of subvisible particles (10 and 25 μm) across different formulations, neither versus time or storage temperature (data not shown). There was also no dramatic change in the number of subvisible particles (10 and 25 μm) after agitation or freeze thaw cycling stress (data not shown).

In conclusion, acetate buffered formulations provided optimal physical and chemical stability relative to histidine. Subvisible particle counts were highly sensitive to the identity of buffer species and pH, with acetate formulations having the lower pH (4.7 and 5.0) generating the lowest particle counts. Turbidity and opalescence were also pH-dependent; formulations were much less opalescent at lower pH acetate conditions, indicating less attractive protein-protein interactions at lower pH. Less opalescent solutions were also shown to have shorter processing times during UFDF operations, which are important manufacturing considerations need for generating high concentration drug substance.

The addition of PS80 to the final formulation was needed to mitigate aggregation and especially particulation risks, which were shown to be accelerated by interfacial stresses such as agitation or freezing/thawing cycling. PS80 was also effective at mitigating or reducing protein adsorption to IV infusion components (e.g., IV bags). PS80 concentrations≥0.05% provided optimal stability.

Significant oxidation and moderate aggregation levels in metal-spiked samples was observed under accelerated storage conditions. Metal chelators were added to the formulation to provide protection from potential metal-induced protein and excipient degradation that could occur. 10 μM EDTA and 10 μM DTPA provided comparable levels of protection and resulted in similar changes in HMWS, oxidation, and PS80 concentrations.

8% sucrose, which is roughly equivalent to 80 mg/ml, was found to be a ratio suitable to protect both the DP and DS concentrations of 50 mg/ml and 150 mg/ml, respectively.

The results from these stability studies demonstrate the robustness of the target formulation to stabilize frozen DS as well as a liquid DP under a variety of the storage and other stress conditions.

### Example 7: Alternate Formulation of Ab1 [Powder for Solution for Infusion]

A further exemplary Ab1 formulation is a sterile lyophilized product. The drug product is filled in a USP Type 1 borosilicate glass vial, 10.3 mL/vial with 0.3 mL of overage. The vial is stoppered with a siliconized gray butyl rubber stopper and sealed with an aluminum seal and a flip-off cap. Table 8 provides information on the exemplary composition of Ab1 drug product.

For administration, each vial is reconstituted with 9.7 mL of Water for Injection to result in a protein concentration of 25 mg/mL, in an aqueous solution containing 10 mM L-histidine, 2% (w/v) sucrose, 3.5% (w/v) mannitol, 10 mM L-methionine, 0.01% (w/v) polysorbate 80, pH 6.0 at 22° C. (Table 8).

Excipient screening showed that 2% sucrose reduced the formation of aggregates during freezing and thawing. The bulking agent mannitol did not significantly affect protein stability, but helped produce an elegant lyophilized cake.

TABLE 8
Alternative Exemplary Formulation for Ab1

Drug product 25 mg/mL
10 mM L-Histidine HCL
2% (w/v) sucrose
3.5% (w/v) mannitol
10 mM L-methionine
0.01% PS80
pH 0.6

1. A pharmaceutical composition, wherein the composition is an aqueous liquid solution comprising:
20-200 mg/ml anti-TGFβ antibody, wherein the antibody comprises a heavy chain variable domain (V_{H}) amino acid sequence corresponding to residues 1-120 of SEQ ID NO:1 and a light chain variable domain (V_{L}) amino acid sequence corresponding to residues 1-108 of SEQ ID NO:2,
10-50 mM acetate, optionally 25 mM acetate, and
5-15% w/v sucrose, optionally 8% w/v sucrose,
wherein the solution has a pH of 5.0±0.2 or 5.0±0.3.

2. The composition of claim 1, wherein the antibody comprises a heavy chain amino acid sequence set forth in SEQ ID NO:1 (with or without the C-terminal lysine) and a light chain amino acid sequence set forth in SEQ ID NO:2.

3. The composition of claim 1, further comprising a surfactant.

4. The composition of claim 3, wherein the surfactant is polysorbate, optionally polysorbate 80 (PS80).

5. The composition of claim 4, wherein PS80 is at a concentration of 0.01-0.10% w/v, optionally 0.06% w/v.

6. The composition of claim 1, wherein the anti-TGFβ antibody is at a concentration of 40-180 mg/ml, optionally 50 mg/ml or 150 mg/ml.

7. The composition of claim 1, further comprising a chelating agent, optionally selected from EDTA and DPTA.

8. The composition of claim 7, wherein the chelating agent is at a concentration of 0 to 20 μM, optionally 10 μM.

9. The composition of claim 1, wherein the aqueous liquid solution has a pH of 4.7-5.3.

10. The composition of claim 1, comprising:
50 mg/ml, 75 mg/ml, or 150 mg/ml anti-TGFβ antibody,
25 mM acetate,
10 μM EDTA,
0.06% PS80, and
8% w/v sucrose,
with a pH of 5.0±0.3.

11. The composition of claim 10, wherein the antibody comprises a heavy chain amino acid sequence set forth in SEQ ID NO:1 and a light chain amino acid sequence set forth in SEQ ID NO:2.

12. An article of manufacture, comprising a vial and instructions for use, wherein the vial contains about 16 ml of the composition of claim 11.

13. A method of treating cancer in a patient in need thereof, comprising administering to the patient a therapeutically effective amount of the composition of claim 1.

14. The method of claim 13, wherein the method further comprises administering an additional anti-cancer therapeutic.

15. The method of claim 13, wherein the composition is administered intravenously at a dose of 5 mg/kg or 15 mg/kg, optionally biweekly.
